(19) United States
(12) Patent Application Publication
(10) Pub. No.: US 2024/0177817 A1
(43) Pub. Date: May 30, 2024

(54) VITAL SIGN DATA MANAGEMENT SYSTEM AND METHOD

(71) Applicant: Micronet Co. Ltd., Sapporo-shi Hokkaido (JP)

(72) Inventor: Kazuhiko ITO, Chuo-ku, Sapporo-shi Hokkaido (JP)

(21) Appl. No.: 17/758,425

(22) PCT Filed: Dec. 6, 2021
(86) PCT No.: PCT/JP2021/044620

§ 371 Date: Jul. 6, 2022

(30) Foreign Application Priority Data
May 28, 2021 (JP) 2021-090214

(51) Int. Cl. G16H 15/00; G06F 21/62; G16H 10/60; G16H 40/67

(52) U.S. Cl. CPC G16H 15/00; G06F 21/6263; G16H 10/60; G16H 40/67; G06F 2221/2151

(57) ABSTRACT

To provide a vital sign data management system and method that share vital sign data of children in real time between a facility side such as a nursery school, a daycare center, or a school, and a household side, and make the data useful in the healthcare of children.

The above-described problem is solved by a vital sign data management system that, when vital signs of a family member are measured and recorded on a household-side communication terminal (1), makes it possible to transmit the vital signs from the communication terminal (1) to a data server (3), which is external, via an Internet (100), register the vital signs in a vital sign data field of a record having a member ID of the family in a household-side database (DB1) on the data server (3) and, at the same time, write the vital signs into a record having the member ID of the same person in a facility-side database (DB2) by a record address KEY notified from the facility side, and share and check the vital sign data of the child and the student between the household side and the facility side in real time.

### FIELD OF THE INVENTION

The present invention relates to a vital sign data management system and method of periodically measuring a vital sign, such as a temperature, of a child such as an infant, a preschooler, or a student at a household, for example, transmitting the vital sign data online to a database for a facility such as a nursery school, a daycare center, a preschool, or a school, registering and managing the data in the database, and conversely individually notifying a guardian of the vital sign data of each child measured on the facility side.

### DESCRIPTION OF THE BACKGROUND ART

In the past, there have been provided in various forms of systems that utilize a mobile communication terminal and the Internet to record, manage, and view various vital sign data on a database of a web server in combination with a cloud service connected to the Internet.

For example, there are mechanisms widely used in temperature data management by, specifically, a basal thermometer, that save recorded data of a temperature measurement result in a database, visualize changes over time by a graph or the like, assist in identification of a state of health and, in a case in which the measured temperature data exceeds a certain threshold value, make it possible to easily realize an operation such as issuance of an alert. Similarly, various devices such as a blood pressure measuring device that records blood pressure measurement results daily as data and identifies a state of health from the changes over time are already widely used as effective mechanisms.

On the other hand, as an application other than continuous measurement, there is a case example of temperature measurement due to the coronavirus crises in recent years. The purpose of this measurement is to maintain social safety by identifying persons with a fever and recording the temperature measurement data at that point in time. In this case, the technique of notification of the temperature measurement data is prioritized over recording the measurement data, and is a technique of displaying an alert and simultaneously notifying other communication terminals via the Internet if the temperature is high at the time of measurement, and managing and monitoring the measurement data.

In addition to healthcare, case examples of recording and managing such various vital signs include use in stress checks or the like as part of safety management when working in specific environments and attendance management in a company and, with the development of mobile devices and the development of the Internet of Things (IoT), the Internet, AI technology, and the like in recent years, effective methods of utilizing vital signs in cooperation with these technologies have been proposed. For example, Patent Document 1 (Japanese Laid-Open Patent Application No. 2013-218699) discloses a technique of automatically measuring the vital data of an individual by a sensor attached to an arm of the individual, transmitting the data to a remote server via the Internet through a mobile communication terminal, registering the data in a database managed by the server, and centrally managing the health of each individual on the server side.

Due to the influence of the coronavirus crises in recent years, there is an increasing number of organizations that measure vital signs, especially in medical facilities, children facilities, companies, institutions, and the like, and utilize the data for the healthcare and infection prevention of children, students, and employees. Amid such a reality, temperature measurement is being actively adopted as one of the vital signs in particular. In the case of this temperature measurement, rather than simply monitoring those with a fever, there is an increasing number of cases in which vital signs are recorded and managed with a focus on temperature measurement as part of daily physical condition management. Even in a school or the like, there is an increasing number of cases in which temperature measurements are taken every morning.

Furthermore, in the case of a childcare facility, in order to care for and manage the physical condition and health of children on a daily basis, recording and managing vital sign data is effective in terms of service and in terms of practice. For example, instead of expressing the state of health and the like of a child during the day with objective sentences while entrusted with a child in a childcare facility, if there is a mechanism that carries out a direct evaluation by vital sign data and makes the information sharable with a guardian side in real time, it is possible to provide the guardian side with a sense of security and save the facility side the trouble of notifying the guardian each time an evaluation is made, solving as a whole problems such as proper identification of the state of health of the child and unexpected delays in response.

Further, when the temperatures of children are to be measured every morning at a school or the like, the tasks of submitting the result of the temperature measurement taken at each household of the children to the school as a memo or the like and collecting the results every day at the school arise. In such a case as well, if there is a mechanism that can notify the school side of the measurement result at each household in real time, the problem of the inefficient and uncertain task of measuring and recording temperatures on the school side can be solved. For example, Patent Document 2 (Japanese Laid-Open Patent Application No. 2019-046242) has been proposed.

As such a case of measuring, registering, and managing the temperature data of a large number of individuals, consider the case of collecting the temperature data measured at each household every morning at an elementary school. The problem emphasized here is not the action to be taken in response to a fever, but how to compile the measurement results. With the spread of the Internet and mobile communication terminals, it is easy to imagine a technique of transmitting temperature measurement data to the school by e-mail, a social networking service (SNS), or the like. However, in this case, there are many problems related to reliability, such as format problems and non-transmission problems, and these problems can be easily solved by creating a dedicated application for recording vital sign data. However, in that case, problems related to the database arise.

In a case in which the elementary school side holds and manages the vital sign data of all students on a data server, there are naturally problems related to security aspects, and it is necessary to control accounts and consider data access permissions and the like. For access to this data server, it is technically possible to create an account for each household with limited data access permissions for each household. Nevertheless, this is very inefficient and, all the more, results in poor management efficiency. A login account must be generated for each household, passwords must also be managed and, if the entity is a school, there are class changes, transfers into and out of the school, and the like every year. Thus, in this case, the labor burden of the task of managing the corresponding household accounts on the school side is greater than the burden of the original task of managing the vital sign data.

Further, assume that the temperatures of children are measured in the morning, at noon, and in the evening at a childcare facility. In this case, the temperatures of the children are recorded in the database of the childcare facility each time. At that time, objective comments or the like can also be recorded. If the data is registered on a data server that can be connected via the Internet, it is also possible to view the data via the Internet. In this case as well, it is necessary to manage account permissions for viewing. The side providing the service also bears the burden of managing account permissions in accordance with a privacy policy. In this case, if the contact information of the guardian is known, it is possible to easily unilaterally notify the guardian of the measurement results at the childcare facility, but conversely, it is not possible to post a result from the household to the childcare facility, and thus the communication remains one way. In this case as well, as in the case example of the elementary school, a method of contacting the childcare facility with the data measured at the household in the morning and, in a case in which the measurement data at noon and in the evening needs to be sharable with each household, taking such action at the childcare facility is required.

### PRIOR ART DOCUMENTS

### Patent Documents

Patent Document 1: Japanese Laid-Open Patent Application No. 2013-218699
Patent Document 2: Japanese Laid-Open Patent Application No. 2019-046242

### SUMMARY OF THE INVENTION

### Problems to be Solved by the Invention

The present invention has been made in view of technical problems in the related art such as described above, and an object of the present invention is to provide a vital sign data management system and method that allows proper information sharing in real time with alleviation of problems of privacy that may occur when recording vital sign data obtained by vital sign measurement in a database of a data server connected via the Internet and managing and viewing the vital sign data in the database, and restrictions when accessing the database of the data server from communication terminals of each facility and each household.

### Means for Solving the Problems

One aspect of the present invention is a vital sign data management system comprising a plurality of first communication terminals respectively provided in a plurality of household sides, a second communication terminal on a facility side, and a vital sign data management server connected to the plurality of first communication terminals and the second communication terminal via an Internet.

The plurality of first communication terminals are each installed with a vital sign data management application and configured to receive input of a group name in units of household and a member name of each member of the household, and generate IDs unique to each of the member names to notify the vital sign data management server thereof, associate vital sign data measured or manually input for each of the member names with the ID unique to the member name, and transmit, to the vital sign data management server via the Internet, the vital sign data along with a facility-side record address informed by the facility side.

The second communication terminal is installed with the vital sign data management application and configured to receive input of one or a plurality of group names on the facility side and each member name belonging to each group, and generate IDs unique to the member names to notify the vital sign data management server thereof, issue, for each of the member names, the facility-side record address unique and corresponding to the ID unique to the member name, associate vital sign data measured or manually input for each of the member names with the ID unique to the member name, and transmit the vital sign data to the vital sign data management server via the Internet.

The vital sign data management server includes a first database for a household side and a second database for the facility side and is configured to assign a unique first database ID to the first database, assign a unique second database ID to the second database, create a record for each of the member names on the household side, associate the IDs unique to each of the member names with the records, and register, for each of the records, the facility-side record address associated with the ID unique to each of the member names, in the first database, create a record for each of the member names on the facility side, associate the IDs unique to each of the member names with the records, and register, for each of the records, the facility-side record address associated with the ID unique to each of the member names, in the second database, record vital sign data, transmitted with specification of the ID unique to each of the member names from the first communication terminals to the first database, in a vital sign data field in the record having the ID unique to each of the member names in the first database, and when the vital sign data is recorded in the vital sign data field of the first database, use the facility-side record address transmitted along with the new vital sign data from the first communication terminal as a key to identify the ID unique for the member name of the facility-side record address registered in the second database, write the same data into the vital sign data field of the record having the ID, and notify the second communication terminal of data writing.

In the vital sign data management system of the above-described invention, the vital sign data management server, when identifying the ID unique for the member name of the facility-side record address registered in the second database and writing the same data into the vital sign data field of the record having the ID, can prohibit, when a time stamp of the data is identical to a time stamp of the vital sign data previously written, the writing of the vital sign data subsequently generated.

Further, in the vital sign data management system of the above-described invention, the second communication terminal is used on the facility side, such as in a nursery school, a daycare center, a preschool, or a school entrusted with children, and the group name registered in the second database may be used as a name of a class, and the member names may be used as names of children belonging to the class.

Another aspect of the present invention is a vital sign data management system comprising a plurality of first communication terminals respectively provided in a plurality of household sides, a second communication terminal on a facility side, and a vital sign data management server connected to the plurality of first communication terminals and the second communication terminal via the Internet.

The plurality of first communication terminals are each installed with a vital sign data management application and configured to receive input of a group name in units of household and a member name of each member of the household, and generate IDs unique to each of the member names to notify the vital sign data management server thereof, issue, for each of the member names, a household-side record address unique and corresponding to the ID unique to the member name, associate vital sign data measured or manually input for each of the member names with the ID unique to the member name, and transmit, to the vital sign data management server via the Internet, the vital sign data along with a facility-side record address informed by the facility side.

The second communication terminal is installed with the vital sign data management application and configured to receive input of one or a plurality of group names in a facility and each member name belonging to each group, and generate IDs unique to each of the member names to notify the vital sign data management server thereof, issue, for each of the member names, the facility-side record address unique and corresponding to the ID unique to the member name, associate vital sign data measured or manually input for each of the member names with the ID unique to the member name, and transmit, to the vital sign data management server via the Internet, the vital sign data with the household-side record address informed by the household side added thereto.

The vital sign data management server includes a first database for the household side and a second database for the facility side and is configured to assign a unique first database ID to the first database, assign a unique second database ID to the second database, create a record for each of the member names on the household side, associate the IDs unique to each of the member names with the records, and register, for each of the records, the household-side record address and the facility-side record address associated with the ID unique to each of the member names, in the first database, create a record for each of the member names on the facility side, associate the IDs unique to each of the member names with the records, and register, for each of the records, the household-side record address and the facility-side record address associated with the ID unique to each of the member names, in the second database, record vital sign data, transmitted with specification of the ID unique to each of the member names from the first communication terminal to the first database, in a vital sign data field in the record having the ID unique to each of the member names in the first database, when the vital sign data is recorded in the vital sign data field of the first database, use the facility-side record address transmitted along with the vital sign data from the first communication terminal as a key to identify the ID unique for the member name of the facility-side record address registered in the second database, write the same data into the vital sign data field of the record having the ID, and notify the second communication terminal of data writing, and when the vital sign data is newly recorded in the vital sign data field of the second database, use the household-side record address transmitted along with the new vital sign data from the second communication terminal as a key to identify the ID unique for the member name of the household-side record address registered in the first database, write the same data into the vital sign data field of the record having the ID, and notify the first communication terminal of data writing.

In the vital sign data management system of the above-described invention, the vital sign data management server, when identifying the ID unique for the member name of the household-side record address registered in the first database and writing the same data into the vital sign data field of the record having the ID, can prohibit, when a time stamp of the data is identical to a time stamp of the vital sign data previously written, the writing of the vital sign data subsequently generated, and when identifying the ID unique for the member name of the facility-side record address registered in the second database and writing the same data into the vital sign data field of the record having the ID, can prohibit, when a time stamp of the data is identical to a time stamp of the vital sign data previously written, the writing of the vital sign data subsequently generated.

Further, in the vital sign data management system of the above-described invention, the second communication terminal is used on the facility side, such as in a nursery entrusted with infants or a daycare center entrusted with preschoolers, and the group name registered in the second database may be used as a name of a class of a facility such as a nursery school or a daycare center, and the member names may be used as names of infants entrusted to the nursery school or names of preschoolers entrusted to the daycare center.

Further, in the vital sign data management system of the above-described invention, text data and image data may be recordable in the vital sign data field in each record of the first database and the second database.

Further, another aspect of the present invention is a vital sign data management method used in a vital sign data management system including a plurality of first communication terminals respectively provided on a plurality of household sides, a second communication terminal on a facility side, and a vital sign data management server connected to the plurality of first communication terminals and the second communication terminal via an Internet.

The vital sign data management method comprises, in the plurality of first communication terminals, by execution of a vital sign data management application installed therein, receiving input of a group name in units of household and a member name of each member of the household, and generating IDs unique to each of the member names to notify the vital sign data management server thereof, associating vital sign data measured or manually input for each of the member names with the ID unique to the member name, and transmitting, to the vital sign data management server via the Internet, the vital sign data along with a facility-side record address informed by the facility side,
in the second communication terminal, by execution of the vital sign data management application installed therein, receiving input of one or a plurality of group names on the facility side and each member name belonging to each group, and generating IDs unique to each of the member names to notify the vital sign data management server thereof, issuing, for each of the member names, the facility-side record address unique and corresponding to the ID unique to the member name, associating vital sign data measured or manually input for each of the member names with the ID unique to the member name, and transmitting the vital sign data to the vital sign data management server via the Internet, and
in the vital sign data management server, assigning a unique first database ID to a first database for the household side, assigning a unique second database ID to a second database for the facility side, creating a record for each of the member names on the household side, associating the IDs unique to each of the member names with the records, and registering, for each of the records, the facility-side record address associated with the ID unique to each of the member names, in the first database, creating a record for each of the member names on the facility side, associating the IDs unique to each of the member names with the records, and registering, for each of the records, the facility-side record address associated with the ID unique to each of the member names, in the second database, recording vital sign data, transmitted with specification of the ID unique to each of the member names from the first communication terminal to the first database, in a vital sign data field in the record having the ID unique to each of the member names in the first database, and w % ben the vital sign data is recorded in the vital sign data field of the first database, using the facility-side record address transmitted along with the new vital sign data from the first communication terminal as a key to identify the ID unique for the member name of the facility-side record address registered in the second database, writing the same data into the vital sign data field of the record having the ID, and notifying the second communication terminal of data writing.

In the vital sign data management method of the above-described invention, the vital sign data management server, when identifying the ID unique for the member name of the facility-side record address registered in the second database and writing the same data into the vital sign data field of the record having the ID, can prohibit, when a time stamp of the data is identical to a time stamp of the vital sign data previously written, the writing of the vital sign data subsequently generated.

Further, in the vital sign data management method of the above-described invention, the second communication terminal is used on the facility side, such as in a nursery school, a daycare center, a preschool, or a school entrusted with children, and the group name registered in the second database may be used as a name of a class, and the member names may be used as names of students belonging to the class.

Yet another aspect of the present invention is a vital sign data management method used in a vital sign data management system including a plurality of first communication terminals respectively provided in a plurality of household sides, a second communication terminal on a facility side, and a vital sign data management server connected to the plurality of first communication terminals and the second communication terminal via an Internet.

The vital sign data management method comprising, in the plurality of first communication terminals, by execution of a vital sign data management application installed therein, receiving input of a group name in units of household and a member name of each member of the household, and generating IDs unique to the member names to notify the vital sign data management server thereof, issuing, for each of the member names, a household-side record address unique and corresponding to the ID unique to the member name, associating vital sign data measured or manually input for each of the member names with the ID unique to the member name, and transmitting, to the vital sign data management server via the Internet, the vital sign data along with a facility-side record address informed by the facility side,
in the second communication terminal, by execution of the vital sign data management application installed therein, receiving input of one or a plurality of group names in a facility and each member name belonging to each group, and generating IDs unique to the member names to notify the vital sign data management server thereof, issuing, for each of member, the facility-side record address unique and corresponding to the ID unique to the member name, associating vital sign data measured or manually input for each of the member names with the ID unique to the member name, and transmitting, to the vital sign data management server via the Internet, the vital sign data with the household-side record address informed by the household side added thereto, and
in the vital sign data management server, assigning a unique first database ID to the first database for the household side, assigning a unique second database ID to the second database for the facility side, creating a record for each of the member names on the household side, associating the IDs unique to each of the member names with the records, and registering, for each of the records, the household-side record address and the facility-side record address associated with the ID unique to each of the member names, in the first database, creating a record for each of the member names on the facility side, associating the IDs unique to each of the member names with the records, and registering, for each of the records, the household-side record address and the facility-side record address associated with the ID unique to each of the member names, in the second database, recording vital sign data, transmitted with specification of the ID unique to each of the member names from the first communication terminal to the first database, in a vital sign data field in the record having the ID unique to each of the member names in the first database, when the vital sign data is recorded in the vital sign data field of the first database, using the facility-side record address transmitted along with the vital sign data from the first communication terminal as a key to identify the ID unique for the member name of the facility-side record address registered in the second database, writing the same data into the vital sign data field of the record having the ID, and notifying the second communication terminal of data writing, and when the vital sign data is recorded in the vital sign data field of the second database, using the household-side record address transmitted along with the vital sign data from the second communication terminal as a key to identify the ID unique for the member name of the household-side record address registered in the first database, writing the same data into the vital sign data field of the record having the ID, and notifying the first communication terminal of data writing.

In the vital sign data management method of the above-described invention, the vital sign data management server, when identifying the ID unique for the member name of the household-side record address registered in the first database and writing the same data into the vital sign data field of the record having the ID, can prohibit, when a time stamp of the data is identical to a time stamp of the vital sign data previously written, the writing of the vital sign data subsequently generated, and when identifying the ID unique for the member name of the facility-side record address registered in the second database and writing the same data into the vital sign data field of the record having the ID, can prohibit, when a time stamp of the data is identical to a time stamp of the vital sign data previously written, the writing of the vital sign data subsequently generated.

Further, in the vital sign data management method of the above-described invention, the second communication terminal is used on the facility side, such as in a nursery entrusted with infants or a daycare center entrusted with preschoolers, and the group name registered in the second database may be used as a name of a class of a facility such as a nursery school or a daycare center, and the member names may be used as names of infants entrusted to the nursery school or names of preschoolers entrusted to the daycare center.

Furthermore, in the vital sign data management method of the above-described invention, text data and image data may be recordable in the vital sign data field in each record of the first database and the second database.

In the above-described invention, the facility-side record address is constituted by a member ID uniquely generated in the facility-side database and a unique ID of the facility-side database, which are notified from the facility side. Then, this record address is encrypted when communicated by the facility side, and changed to easily input notation called a “notification key.” It should be noted that notation conversion by this encryption is often used in general, and can also serve as a measure to prevent input errors such as the letter O and the number 0 (zero) not being used or the notation being unified to all uppercase letters.

### Effect of the Invention

According to the vital sign data management system and method of the present invention, when a vital sign data management application is started and vital signs of a member of a family are measured and data of the vital signs are recorded on a communication terminal on a household side, it is possible to transmit the vital sign data from the communication terminal to a data server, which is external, via the Internet, register the vital sign data in a vital sign data field of a record having an ID of the member of the family in a household-side database on the data server and, at the same time, when a record address notified from the facility side is linked with the ID of the member of the family, not only transmit the vital sign data to the data server and register the vital sign data in a record having the corresponding ID in the household-side database, but also use the record address as a key to notify and write the vital sign data into a record having the member ID on the facility side having a common record address in the facility-side database and, by using a communication terminal on the facility side to access the facility-side database, read and check, from the record having the corresponding ID in the facility-side database, the vital sign data of the member of the family newly written from the communication terminal on the household side. On the other hand, the records of the members of the family in the family-side database cannot be directly accessed from the facility side, and thus the records in the household-side database cannot be falsified or deleted from the facility side, and security and reliability can be maintained.

Further, according to the vital sign data management system and method of the present invention, when a vital sign data management application is started and vital signs of a member on a facility side are measured and vital sign data are recorded on a communication terminal on the facility side, it is possible to transmit the vital sign data from the communication terminal to a data server, which is external, via the Internet, register the vital sign data in a vital sign data field of a record having an ID of the member in a facility-side database on the data server and, at the same time, use the record address as a key to notify and write the vital sign data into a record having the member ID on the family side having a common record address in the family-side database and, by using a communication terminal on the family side to access the family-side database, read and check, from the record having the corresponding ID in the family-side database, the vital sign data of the member of the facility newly written from the communication terminal on the facility side. On the other hand, the records of the members of the facility in the facility-side database cannot be directly accessed from the family side, and thus the records in the facility-side database cannot be falsified or deleted from the family side, and security and reliability can be maintained.

### BRIEF DESCRIPTION OF THE DRAWINGS

FIG. 1 is a hardware configuration view of a vital sign data management system of one embodiment of the present invention.

FIG. 2 is a block diagram illustrating a functional configuration of a communication terminal and a non-contact thermometer in the vital sign data management system of the above-described embodiment.

FIG. 3 is a block diagram illustrating a functional configuration of a data server in the vital sign data management system of the above-described embodiment.

FIG. 4 is an explanatory view illustrating data management operations of a first database and a second database by a data server in the vital sign data management system of the above-described embodiment.

FIG. 5 is a flowchart of account registration processing on a household side by a household-side communication terminal in the vital sign data management system of the above-described embodiment.

FIG. 6 is a flowchart of account registration processing on a facility side by a facility-side communication terminal in the vital sign data management system of the above-described embodiment.

FIG. 7 is a flowchart of measurement and transmission processing of vital sign data of a family member by the household-side communication terminal in the vital sign data management system of the above-described embodiment.

FIG. 8 is a flowchart of registration processing of vital sign data, transmitted from the household-side communication terminal by the data server, into a first database in the vital sign data management system of the above-described embodiment.

FIG. 9 is a flowchart of verification processing of newly registered vital sign data of a child member by the facility-side communication terminal in the vital sign data management system of the above-described embodiment.

FIG. 10 is a flowchart of measurement and transmission processing of vital sign data for each class and child by the facility-side communication terminal in the vital sign data management system of the above-described embodiment.

FIG. 11 is a flowchart of registration processing of vital sign data, transmitted from the facility-side communication terminal by the data server, into a second database in the vital sign data management system of the above-described embodiment.

FIG. 12 is a flowchart of verification processing of newly registered vital sign data of a family member by the household-side communication terminal in the vital sign data management system of the above-described embodiment.

FIG. 13 is a table showing a correspondence relationship between member IDs and records in the first database in the vital sign data management system of the above-described embodiment.

FIG. 14 is a table showing a correspondence relationship between member names of each household and record addresses registered in the first database in the vital sign data management system of the above-described embodiment.

FIG. 15 is an explanatory view showing registered contents of a data field in one record in the first database in the vital sign data management system of the above-described embodiment.

FIG. 16 is an explanatory view of one of the record addresses used in the vital sign data management system of the above-described embodiment.

FIG. 17 is a table showing a correspondence relationship between database IDs, unique IDs of members, and records in the first database in the vital sign data management system of the above-described embodiment.

FIG. 18 is an explanatory view illustrating a data flow when vital sign data is uploaded from a communication terminal on the household side by utilizing a facility-side record address in the vital sign data management system of the above-described embodiment.

FIG. 19 is an explanatory view illustrating a data flow when data of a first database is modified from a communication terminal on the household side in the vital sign data management system of the above-described embodiment.

FIG. 20 is an explanatory view illustrating a data flow when vital sign data is uploaded from communication terminals on the household side and the facility side by respectively utilizing a facility-side record address and a household-side record address in the vital sign data management system of the above-described embodiment.

FIG. 21 is an explanatory view illustrating a data flow when a school side is notified of the vital sign data of a child from the household side in the vital sign data management system of the above-described embodiment.

FIG. 22 is an explanatory view illustrating a data flow when each household side is notified of the vital sign data of a child from the facility side in the vital sign data management system of the above-described embodiment.

### DESCRIPTION OF THE PREFERRED EMBODIMENTS

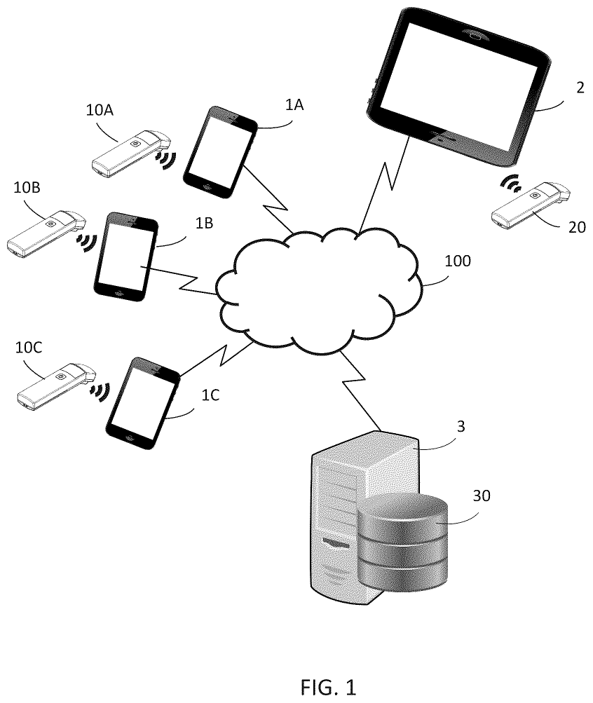

Hereinafter, embodiments of the present invention will be described in detail with reference to the drawings. As illustrated in FIG. 1, a vital sign data management system of an embodiment is constituted by a household-side communication terminal 1 used by a registered user, a facility-side communication terminal 2 used by a facility side entrusted with many children, such as a nursery school, a daycare center, a preschool, or a school, a data server (computer) 3 that performs management processing such as registering, deleting, accumulating, and updating vital sign data, and a database device 30 serving as a storage device managed by the data server 3. The household-side communication terminal 1, the facility-side communication terminal 2, and the data server 3 are connected via an Internet 100.

In the following, description will be made using temperature as a typical example of a vital sign. A thermometer 10 that measures the temperature of a person in a non-contact manner is provided in each household A, B, C, . . . , which are users. The facility is also equipped with a thermometer 20 which is the same as the thermometer 10. Certainly, on the facility side, if the facility is a daycare center or a school, one thermometer 20 may be provided to each class. If the facility is a nursery school of a smaller scale, the device may be shared across classes. Nevertheless, in a case in which the device is shared across classes, the facility name as a whole is registered as a group without the use of class names. Further, in this embodiment, a non-contact thermometer provided with a short-range wireless communication function is exemplified, but in the system of this embodiment, temperature and other vital sign data may be manually input on the communication terminals 1, 2. Furthermore, as illustrated in FIG. 1, in order to distinguish the persons subject to measurement in the descriptions of this specification, reference signs A, B, C, . . . are added to distinguish the individuals.

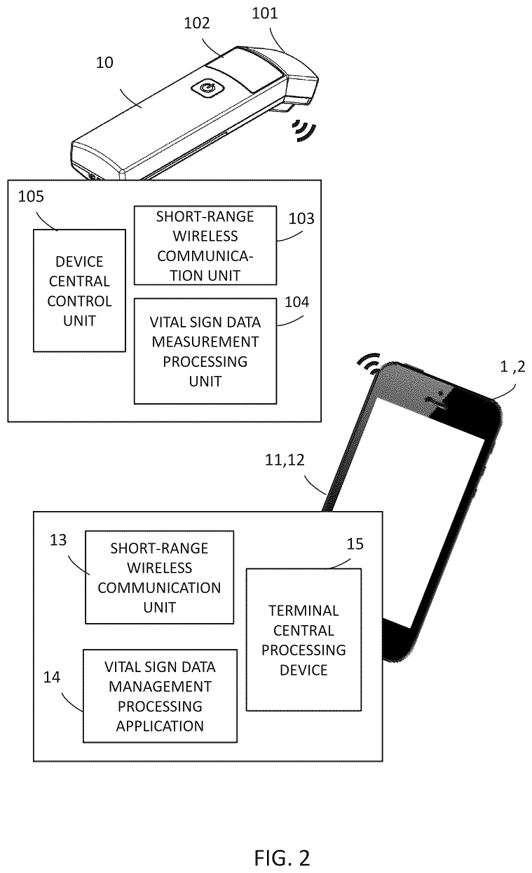

As illustrated in FIG. 2, the thermometers 10, 20 (hereinafter, the reference sign 10 may be used as a representative in descriptions of the thermometer), serving as vital sign data measurement devices, each include an infrared sensor unit 101 that measures temperature in a non-contact manner, a display unit 102 that displays measurement data, a short-range wireless communication unit 103 that controls short-range wireless communication, such as Bluetooth, for example, exchanged with the household-side communication terminal 1 or the facility-side communication terminal 2, and a vital sign data measuring unit 104 that emits infrared rays to the infrared sensor unit 101, calculates a surface temperature from an intensity of reflected infrared rays received by the infrared sensor unit 101, and displays the surface temperature on the display unit 102 as temperature data, and further includes a device central processing unit (CPU) 105 that comprehensively controls the entire operation of the thermometer 10, such as signal output control, display control, and power supply management, for example, and stores and manages measurement and other data.

The household-side communication terminal 1 is a personal computer (PC), a smartphone, a tablet, or the like that performs data management, such as storing, updating, modifying, and deleting vital sign data, transmits vital sign data to an external source through an Internet 100, and receives vital sign data transmitted from an external source through the Internet. This household-side communication terminal 1 includes a display unit 11 that displays various information, an input operation unit 12 that receives input operations of data, a short-range wireless communication unit 13 that performs short-range wireless communication with other devices by using short-range wireless communication means such as Bluetooth, for example, and a vital sign data management processing application 14 specially installed as the system of this embodiment, and further includes a device central processing device 15 that controls the operation of the entire device. The display unit 11 also operates as the input operation unit 12 that receives touch operations. The facility-side communication terminal 2 is basically the same as the household-side communication terminal 1, and has a functional configuration common to that of the household-side communication terminal 1. It should be noted that, in a case in which PCs are used as the communication terminals 1, 2, the display unit 11 is a display, and the input operation unit 12 includes a keyboard and a mouse, and in some cases, further includes a touch panel and a touch-compatible display.

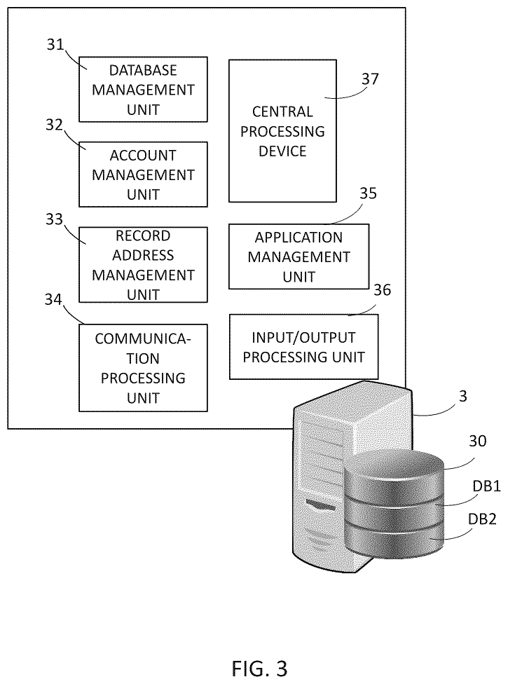

As illustrated in FIG. 3, the data server 3 is provided with the storage device 30 in which a first database DB1 and a second database DB2 are created. The first database DB1 is a household-side database that registers and manages the vital sign data of each person (member) subject to measurement transmitted from each household-side communication terminal 1. The second database DB2 is a facility-side database that registers and manages the vital sign data of each person (member such as a student, an infant, or a preschooler) subject to measurement transmitted from the facility-side communication terminal 2 in a nursery school, a daycare center, a school, or the like. The storage devices 30 that hold the first and second databases DB1, DB2 may be the same or may be different devices. The first and second databases DB1, DB2 are set under administrator permissions by using the administrator-side communication terminal 2 or a PC.

This data server 3 is constituted by a computer, but when divided into related processing functions, is constituted by a database management unit 31 that manages the registration, updating, and deletion of data including the database of the storage device 30, an account management unit 32 that performs account management such as the issuance of various accounts, the setting, verification, and approval of permissions, and access prohibition, a record address management unit 33 that issues record addresses, a communication processing unit 34 that handles communication, an application management unit 35 that manages the creation, registration, updating, and the like of the application, an input/output processing unit 36 that controls input/output such as data input and output display, and a central processing device 37 that comprehensively controls the operation of the entire device.

Next, operation of the vital sign data management system of the above-described configuration will be described. This operation description is also a description of a vital sign data management method executed by the system of this embodiment.

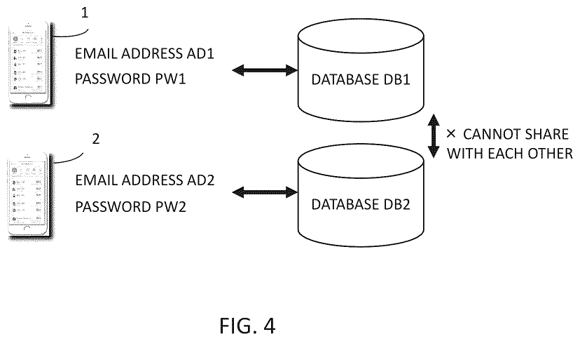

As illustrated in FIG. 4, to access the first database DB1 and the second database DB2, unique accounts are registered. This is managed by a set of generally used email addresses ad1, ad2 and passwords pw1, pw2 and configured so that accounts are not duplicated when the databases are utilized. Here, the first database DB1 and the second database DB2 are independent for each group, such as each household and facility, and the data cannot be mutually accessed from the viewpoint of privacy. For example, the second database DB2 on the facility side cannot be accessed from the household-side communication terminal 1 (that is, an account on the household side), and the first database DB1 on the household side cannot be accessed from the facility-side communication terminal 2 (that is, an account on the facility side). It should be noted that, technically, even if the same communication terminal is used, the terminal can be used on either the household side or the facility side by changing the account at the time of access. Herein, the communication terminals 1, 2 are distinguished for ease of understanding of the explanation.

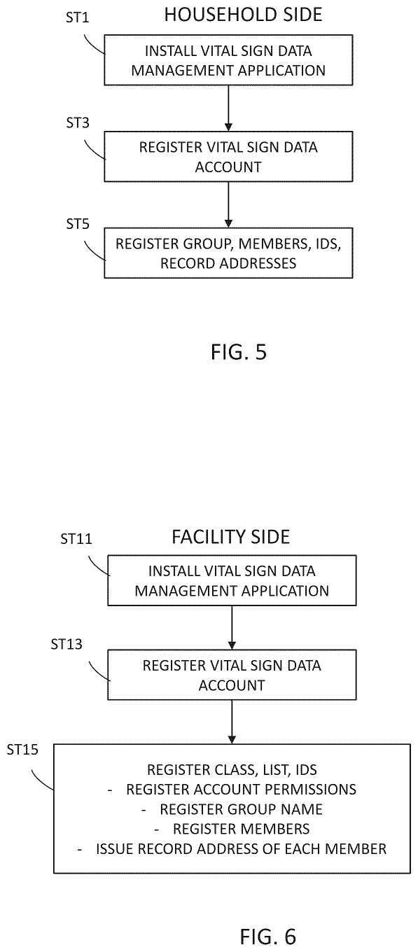

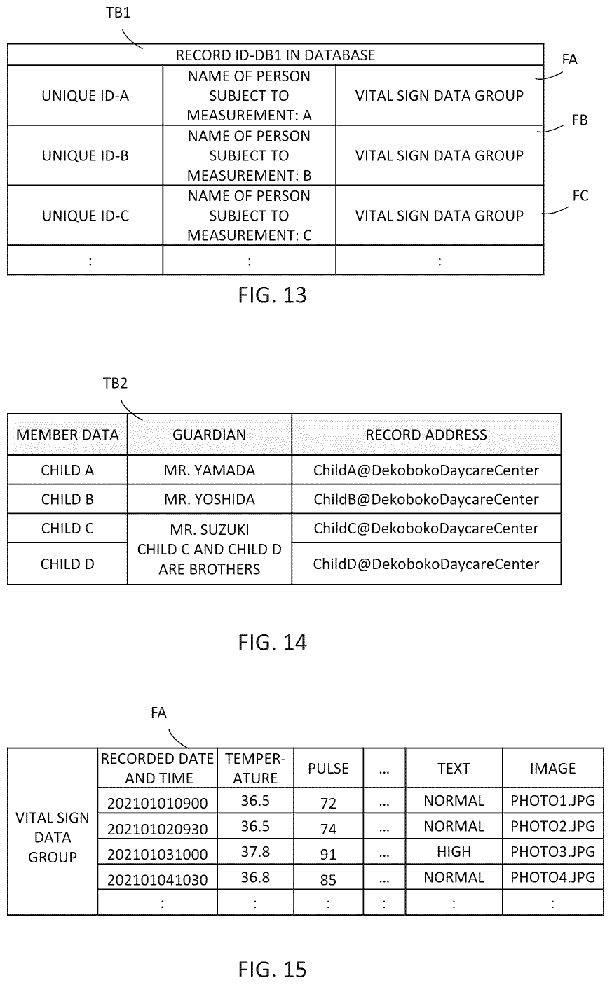

As illustrated in FIG. 5, on the household side, users A, B, C . . . use communication terminals 1A. 1B, 1C . . . to first download and install the vital sign data management application from an application store (step ST1), start this vital sign data management application, and register an individual account in the data server 3 (step ST3). Then, a guardian of each household, as a user, registers the family as a group, and the children entrusted to the nursery school, daycare center, school, or the like as members in the group of the family (step ST5). These are registered in the first database DB1 in the data server 3 with IDs unique to each member, and one record is assigned to each. A list TB1 showing the records in the database in FIG. 13 shows that a unique ID is given and a record is created for each of the members A, B, C, . . . in the first database DB1.

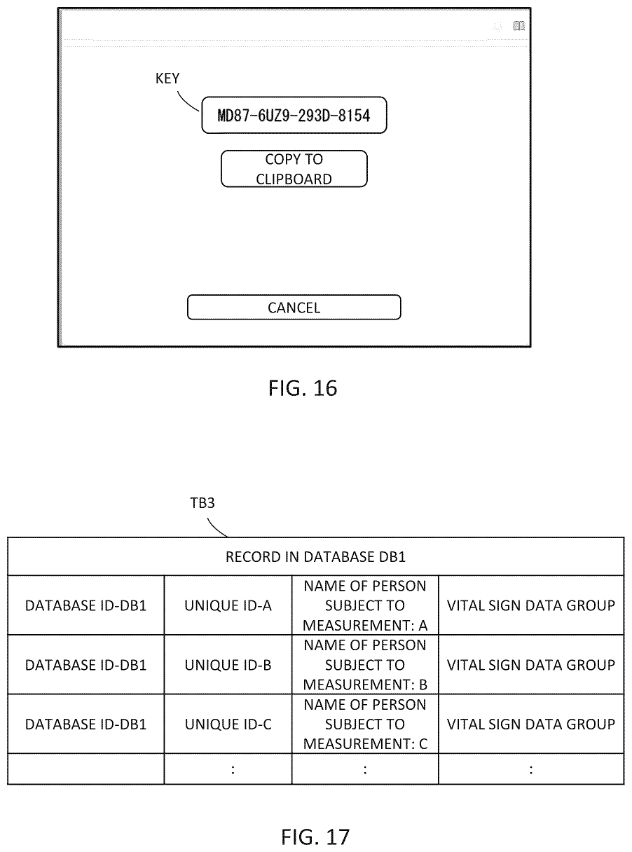

As illustrated in FIG. 6, on the facility side, the communication terminal 2 is used to download and install the vital sign data management application from an application providing site of the data server 3 or the like (step ST11), start this vital sign data management application, and register a user account on the facility side in the data server 3 (step ST13). Furthermore, on the facility side, the communication terminal 2 is used to register account permissions in accordance with responsibility, such as, for example, a group such as a name of a nursery school, a daycare center, a preschool, or a school, a class name, or the like, and members in a list of infants, preschoolers, or children belonging to that group. At this time, a record address KEY such as illustrated in FIG. 16 is simultaneously issued (step S15).

The record address KEY issued on this facility side is unique to each member. Moreover, it is a key indicating permission for notifying only the member having the same unique ID in the first database DB1 corresponding to the member in the second database DB2 of a data update. Accordingly, on the first database DB1 side, data written in the vital sign data field of the record of the record address KEY common to the second database DB2 can be written into the record for which this record address KEY is set.

Types of this account are not particularly limited, but examples include an administrator account, a manager account, and a general user account.

The “administrator account” is an account for an administrator who takes care of and manages the entire system of the facility, and can be registered by a login ID and password. From this administrator account, procedures such as issuance and registration, deletion, and renewal of other accounts can be performed with administrator permissions. On the household side, it is possible for parents of each household to access this system for their child or children, and perform operations such as registration of a group name, registration of each member name of their child or children, and registration of measured vital sign data for each member after registration.

The “manager account” can register login IDs and passwords, provide account explanations, and register a group in charge. Normally, a guardian receives this manager account on the household side, and a teacher in charge of a class receives the manager account on the facility side. Upon accessing the first and second databases DB1, DB2 with a manager account, it is possible to record, edit, and view the vital sign data of each member of the assigned group, and write character data.

The “general user account” can also be acquired by a guardian or a homeroom teacher by registering a login ID and password. With a general user account, it is possible to measure, by the thermometer 10, the temperature of a child of a member assigned as a person subject to measurement, and record the vital sign data in the record having an ID unique to each member defined in the first database DB1 and the second database DB2.

It should be noted that account permissions are not specified in this specification, and are described as permissions that allow the operators of the communication terminal 1 and the communication terminal 2 to perform operations such as group registration, member registration, updating and deletion thereof, and a recording operation of measurement data.

As shown in a list TB2 of FIG. 14, records are assigned and registered one by one with each household defined as a group in the first database DB1 on the household side, and the person(s) subject to measurement in the household, that is, one or a plurality of children who attend the daycare or the school, defined as the members. It should be noted that, for problems such as same gender and same name that may occur in companies, schools, and the like, a list is generated by using unique IDs for the persons subject to measurement in each database so that there are no duplications. For example, if there are a plurality of persons subject to measurement with the name “Hirofumi Ito,” a check is conducted on the data server 3 side and IDs unique to each person, such as “Hirofumi Ito A,” “Hirofumi Ito B,” and “Hirofumi Ito C” are automatically set.

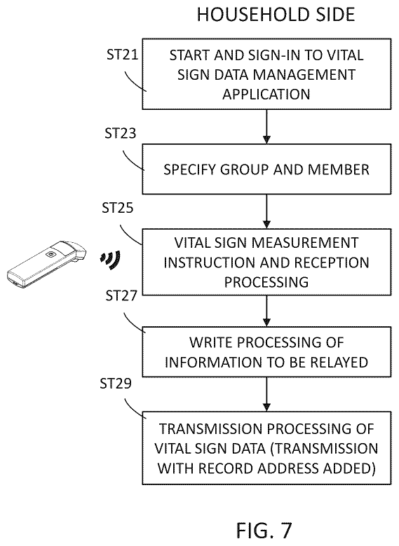

As long as setup and registration are completed on each household side and the facility side, it is possible to record and manage temperature measurement data by using the thermometers 10, 20. On the household side, as illustrated in the flowchart of FIG. 7, the guardian starts the vital sign data management application on the household-side communication terminal 1 to access the database with a general user account (step ST21). Then, the guardian specifies the group to which the household belongs (here, the name of the daycare center that the child is attending) and the member name (here, the name of the child), and waits until short-range wireless communication with the thermometer 10 is established (step ST23). Subsequently, the guardian touches the measurement instruction button to send a measurement instruction to the thermometer 10, and data of the temperature measurement result is received as vital sign data from the thermometer 10 and displayed as the latest data on the screen of the corresponding member of the communication terminal 1 (step ST25). Furthermore, if this child is slightly unwell or further information needs to be relayed, the guardian writes text data (step ST27).

As a result, as shown in table TB1 of FIG. 13, the vital sign data is written into vital sign data fields FA, FB, FC, . . . of records of ID-A, ID-B, ID-C, . . . , respectively, in database ID-DB1 in the first database DB1 for each household. Historical data of a certain period of time is accumulated and saved in the vital sign data fields FA, FB, FC, . . . . At the same time, as in the vital sign data field FA shown in FIG. 15, text data and image data such as information to be relayed can be written into the vital sign data field in each record.

Here, in this embodiment, the database IDs of the first database DB1 and the second database DB2 and the IDs unique to each member are combined to set a “record address,” making it possible to distinguish the databases into which data is to be written and distinguish the individual children. As shown in table TB3 of FIG. 17, the exemplified record address is an address prepared for registering and managing the vital sign data of the children (members) of each household in the first database DB1 for household sides. By adding DB1 as a database ID to the first database DB1 for household sides and further adding the IDs ID-A, ID-B, ID-C, . . . of each child member as the ID of each member, it is possible to uniquely determine which record of the first or the second database DB1 or DB2, is to be used to register and manage the vital sign data of each child member name.

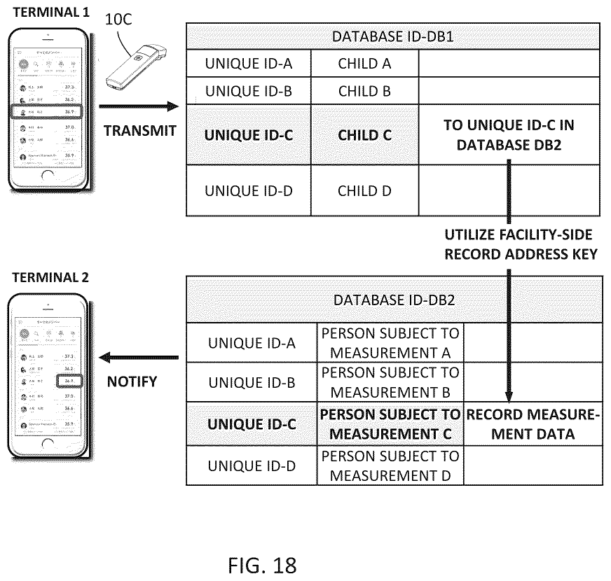

Further, the record address KEY such as illustrated in FIG. 16 mentioned above can be individually generated in correspondence with this record address obtained by combining the database ID and the member ID. This record address KEY is notified from the daycare center to each household by e-mail or other means. Then, the guardians register this record address w % ben registering their child as a member. As a result, as illustrated in FIG. 18, in the data server 3, the record address KEY can be used as a key to identify a member ID having a one-to-one correspondence between the first database DB1 and the second database DB2.

Therefore, in a case in which the facility side, such as a school, is notified of the temperature of a child measured on the household side by the procedure illustrated in the flowchart of FIG. 7, the measured temperature data is used as vital sign data and, with execution of the operation of the transmission instruction, is transmitted to the data server 3 with the record address KEY informed and registered in advance by the facility side added thereto (step ST29).

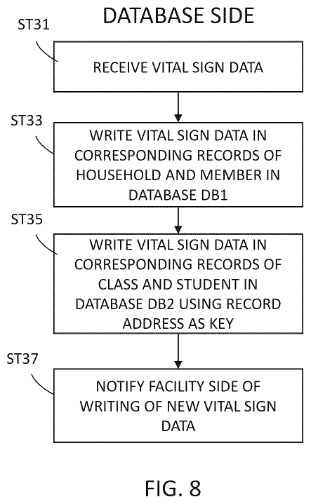

As illustrated in the flowchart of FIG. 8, when the data server 3 side receives the vital sign data from the household side (step ST31), it is determined that the vital sign data should be registered in the first database DB1 on the household side by the account and member ID of the sender, and the received vital sign data is registered in the vital sign data field of the record having the corresponding member ID in the first database (step ST33). Then, the record address KEY is used as a key to determine the unique member ID of the second database DB2 corresponding to the unique member ID of the first database DB1. The unique ID of the first database and the unique ID of the second database DB2 are for the same person. Therefore, the data is written into the vital sign data field of the corresponding record in the second database DB2, and the account on the facility side is notified of this writing (steps ST35, ST37).

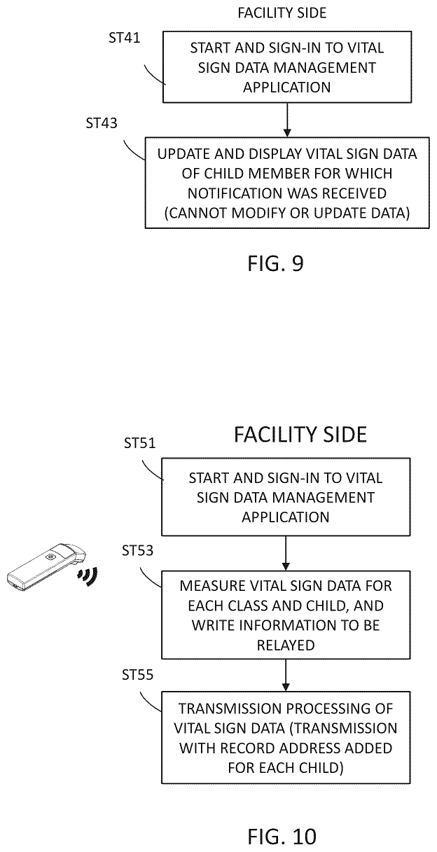

As illustrated in the flowchart of FIG. 9, when the vital sign data management application is started and a view operation is performed, the facility-side communication terminal 2 that received the notification displays that the vital sign data of the child member for which the notification was received has been updated (steps ST41, ST43).

The data registration operation in the data server 3 in response to the processing operations in FIG. 8 and FIG. 9 is illustrated in FIG. 18. That is, in a case in which a notification of a data update by the record address KEY is received, the new vital sign data transmitted from the household side and registered in the first database DB1 is registered in the record having the same record address in the second database DB2.

In this way, a guardian at each household measures and registers the vital sign data of a child entrusted to a daycare center or the like before leaving the house, thereby transmitting the same vital sign data to the second database DB2 of the affiliated group registered in advance, that is, the account of the daycare center, and making it possible to view, on the facility side as well, the vital sign data of the registered child on that day from a record having a member ID unique to the corresponding child in the second database DB2.

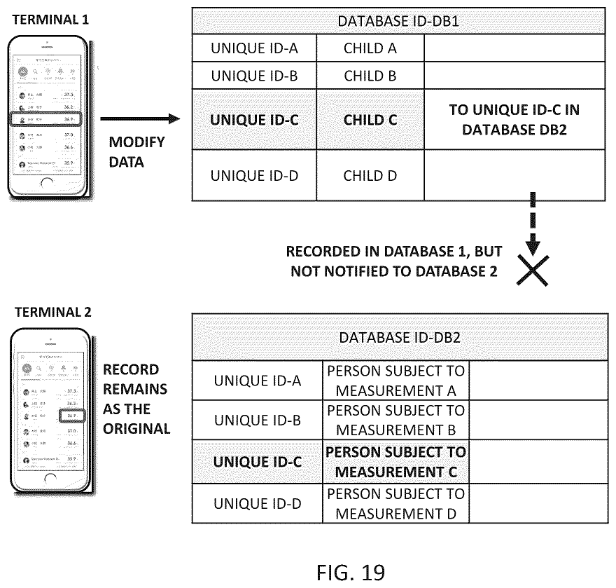

On the other hand, in this case, the facility side can write the vital sign data of the corresponding child into the record having the member ID unique to the corresponding child in the second database DB2, but the written data is not written into the record having the member ID of the corresponding child in the first database DB1 on the household side. Accordingly, data falsification from the facility side can be prevented. Furthermore, as illustrated in FIG. 19, the data of the record having the member ID unique to the child in the first database DB1 on the household side can be modified, but in this case, the modified data is not reflected into the record having the ID of the child in the second database DB2 on the facility side and is not notified from the facility side. In this way, it is possible to prevent data falsification on both sides and thus prevent the occurrence of problems.

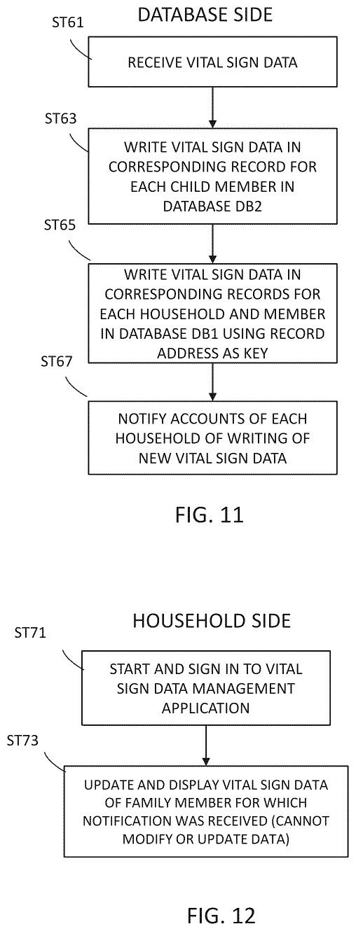

Next, using the flowcharts of FIG. 10, FIG. 11 and FIG. 12, the processing in a case in which the temperature of a child entrusted to a nursery school or daycare center on the facility side is measured and notified to the household side along with information that needs to be relayed will be described. In this case as well, record address KEYs are generated from the facility side along with the member IDs unique to each entrusted child, and are registered upon notification to the household of each child. The facility side measures the temperature of each child by using the thermometer 20 and transmits the temperature to the data server 3 by the communication terminal 2 (steps ST51. ST53). In this case, the data server 3 determines that data has been transmitted from the facility side by the account, and writes the latest vital sign data acquired for each member ID into the second database DB2 on the facility side. Also at that time, the record address KEY issued for each member ID is transmitted with the vital sign data added thereto (step ST55).

As illustrated in FIG. 11, upon receiving the vital sign data for one or each of a plurality of member IDs from the facility-side communication terminal 2, the data server 3 records the vital sign data in the records having each of the member IDs in the second database DB2 on the facility side (steps ST61, ST63). Then, the record address KEY corresponding to each member ID is used as a key to record the same vital sign data written into the second database DB2, for each record of the member ID of the same child in the first database DB1 (step ST65). Subsequently, each account on the household side associated with each corresponding member ID is notified of the presence of newly written vital sign data (step ST67).

As illustrated in FIG. 12, on the household side, when the guardian starts the vital sign data management application on the communication terminal 1 and is informed of a data update notification, the updated data displayed in the record area of the corresponding child can be checked (steps ST71, ST73).

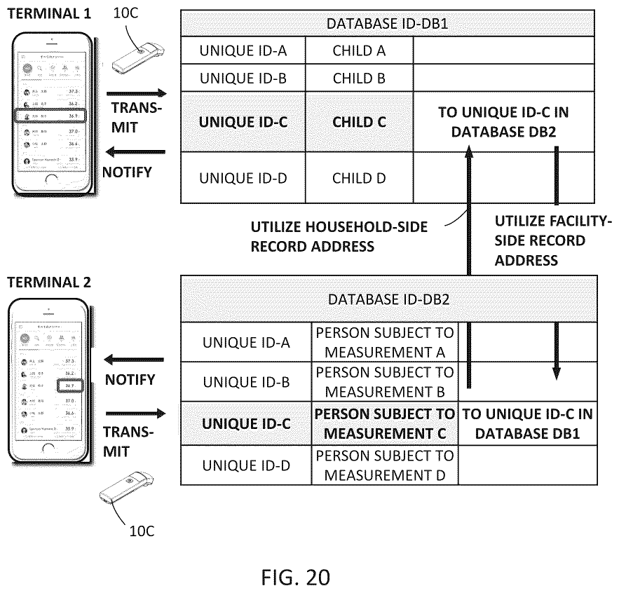

Furthermore, as illustrated in FIG. 20, if the record address KEY is shared between the household side and the facility side, the data of the thermometer 10 acquired by the household-side communication terminal 1 is recorded in the record having the member ID unique to the corresponding child in the first database DB1 on the household side, thereby reflecting the recording in the record having the member ID unique to the corresponding child in the second database DB2 on the facility side. Conversely, the data of the thermometer 20 acquired by the facility-side communication terminal 2 is recorded in the record having the member ID unique to the corresponding child in the second database DB2 on the facility side, thereby reflecting the recording in the record having the member ID unique to the corresponding child in the first database DB1 on the household side. Nevertheless, even though vital sign data newly written from the household side into the first database DB1 is also reflected in the second database DB2 and is notified to the facility side, any modifications or changes made on the facility side to the vital sign data written into the second database DB2 are not reflected in the record having the corresponding member ID in the first database DB1. Conversely, even though vital sign data newly written from the facility side into the second database DB2 is also reflected in the first database DB1 and notified to the household side, any modifications or changes made on the household side to the vital sign data written into the first database DB1 are also not reflected in the record having the corresponding member ID in the second database DB2. As a result, the problem of data falsification is nonexistent, and security can be protected.

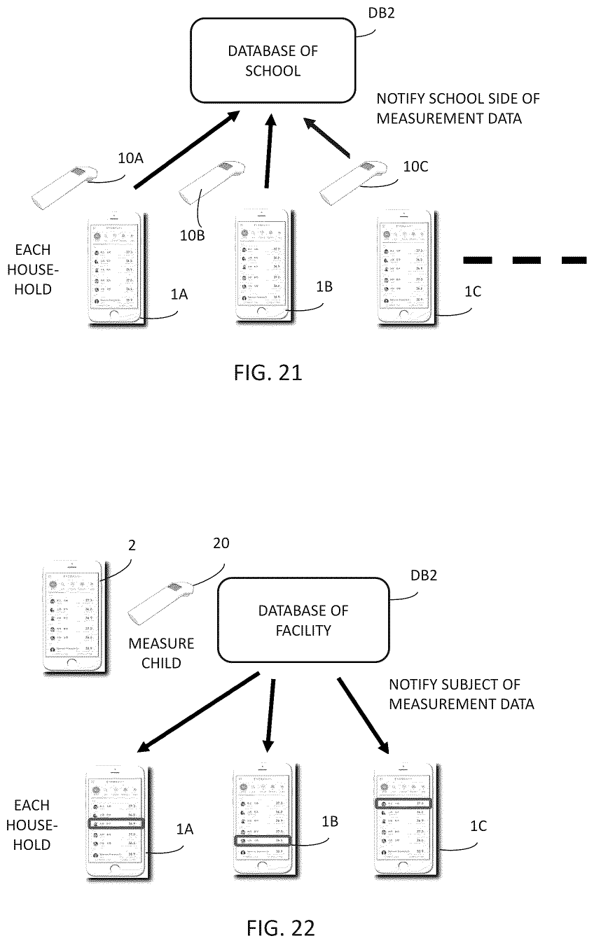

Nowadays, at a school attended by students, there is a desire to collect vital sign data, such as the daily temperature of each student, and reflect the data in healthcare and safety. In such a case, as illustrated in FIG. 21, the temperature data measured before the student leaves the house in the morning can be reflected in the second database DB2 on the school side and checked by the school side as well. Further, as illustrated in FIG. 22, in a facility such as a daycare center, when the vital sign data of each preschooler to be measured on a regular basis is registered in the vital sign data field of the record having the ID of the preschooler in the second database DB2 on the facility side, the data is also reflected in the record having the member ID of the child in the first database DB1 on the household side, making it possible for each household to be informed in detail of the physical condition of the child entrusted to the facility. This can be used in elderly care facilities as well.

Conventionally, in a case in which it is desirable to manage, with highly confidentiality, data including vital sign data and still share information via the Internet, database access permissions and detailed account management are required, resulting in an increasing amount of labor spent on management as the measurement scale increases in size.

On the other hand, in the present invention, the object thereof is to omit complicated account permission management and to be able to easily perform mutual data notification management between a plurality of database DBs by using common record addresses and perform vital sign data management having the advantageous effects of real-time data reflection and data falsification prevention, a data address linking function was developed for the mutual notification function, making it possible to execute system operation by using the same vital sign data management application common to the management side, the terminal side, and the like without distinguishing an application for each side. As a result, the labor required for account management, member registration, data entry, and the like can be significantly reduced. Further, the present invention can contribute to more proper data management by the advantageous effect of preventing the falsification of data in databases, which dates back to the past, and to an improvement in healthcare services centered on vital sign data by being able to transmit necessary notifications to required locations in real time. In addition, both the household side and the facility side can install and use a common application on a communication terminal used at each location, resulting in the advantage that maintenance management and usage explanations of the application are the same.

### INDUSTRIAL APPLICABILITY

The present invention shares vital sign data in real time between a facility side such as a nursery school, a daycare center, a preschool, or a school, and a household side, and is helpful in the healthcare of children. Further, in a case of operation between an elderly care facility and a household, the present invention can be applied to the healthcare of elderly residents and users, and remote monitoring by family.

### DESCRIPTIONS OF REFERENCE NUMERALS

1 (Household-side) communication terminal
2 (Facility-side) communication terminal
3 Data server
10, 20 Non-contact thermometer
11 Display unit
12 Operation unit
13 Short-range wireless communication unit
14 Vital sign data management processing unit
15 Terminal central processing device
30 Storage device
31 Database management unit
32 Account management unit
33 Record address management unit
34 Communication processing unit
35 Application management unit
36 Input/output processing unit
37 Central processing device
100 Internet
101 Infrared sensor unit
102 Display unit
103 Short-range wireless communication unit
104 Vital sign data measurement processing unit
105 Device central control unit
DB1 First database
DB2 Second database

What is claimed is:

1. A vital sign data management system comprising:
a plurality of first communication terminals respectively provided in a plurality of household sides;
a second communication terminal on a facility side; and
a vital sign data management server connected to the plurality of first communication terminals and the second communication terminal via an Internet,
the plurality of first communication terminals each being installed with a vital sign data management application and configured to
receive input of a group name of each household and a member name of each member of the household, and generate IDs unique to each of the member names to notify the vital sign data management server thereof, and
associate vital sign data measured or manually input for each of the member names with the ID unique to the member name, and transmit, to the vital sign data management server via the Internet, the vital sign data along with a facility-side record address informed by the facility side,
the second communication terminal being installed with the vital sign data management application and configured to
receive input of one or a plurality of group names on the facility side and each member name belonging to each group, and generate IDs unique to the member names to notify the vital sign data management server thereof,
issue, for each of the member names, the facility-side record address unique and corresponding to the ID unique to the member name, and
associate vital sign data measured or manually input for each of the member names with the ID unique to the member name, and transmit the vital sign data to the vital sign data management server via the Internet, and
the vital sign data management server including a first database for a household side and a second database for the facility side and being configured to
assign a unique first database ID to the first database,
assign a unique second database ID to the second database,
create a record for each of the member names on the household side, associate the IDs unique to each of the member names with the records, and register, for each of the records, the facility-side record address associated with the ID unique to each of the member names, in the first database,
create a record for each of the member names on the facility side, associate the IDs unique to each of the member names with the records, and register, for each of the records, the facility-side record address associated with the ID unique to each of the member names, in the second database,
record new vital sign data, transmitted with specification of the ID unique to each of the member names from the first communication terminal to the first database, in a vital sign data field in the record having the ID unique to each of the member names in the first database, and
when the new vital sign data is recorded in the vital sign data field of the first database, use the facility-side record address transmitted along with the new vital sign data from the first communication terminal as a key to identify the ID unique for the member name of the facility-side record address registered in the second database, write the same data into the vital sign data field of the record having the ID, and notify the second communication terminal of data writing.

2. The vital sign data management system according to claim 1, wherein
the vital sign data management server, when identifying the ID unique for the member name of the facility-side record address registered in the second database and writing the same data into the vital sign data field of the record having the ID, prohibits, when a time stamp of the data is identical to a time stamp of the vital sign data previously written, the writing of the vital sign data subsequently generated.

3. The vital sign data management system according to claim 2, wherein
the second communication terminal is used on the facility side, such as in a nursery school, a daycare center, a preschool, or a school entrusted with children, and
the group name registered in the second database is a name of a class, and the member names are names of children belonging to the class.

4. The vital sign data management system according to claim 1, wherein
text data and image data can be recorded in the vital sign data field in each record of the first database and the second database.

5. A vital sign data management system comprising:
a plurality of first communication terminals respectively provided in a plurality of household sides;
a second communication terminal on a facility side; and
a vital sign data management server connected to the plurality of first communication terminals and the second communication terminal via an Internet,
the plurality of first communication terminals each being installed with a vital sign data management application and configured to
receive input of a group name of each household and a member name of each member of the household, and generate IDs unique to each of the member names to notify the vital sign data management server thereof,
issue, for each of the member names, a household-side record address unique and corresponding to the ID unique to the member name, and
associate vital sign data measured or manually input for each of the member names with the ID unique to the member name, and transmit the vital sign data to the vital sign data management server via the Internet,
the second communication terminal being installed with the vital sign data management application and configured to
receive input of one or a plurality of group names in a facility and each member name belonging to each group, and generate IDs unique to each of the member names to notify the vital sign data management server thereof, and
associate vital sign data measured or manually input for each of the member names with the ID unique to the member name, and transmit, to the vital sign data management server via the Internet, the vital sign data with the household-side record address informed by the household side added thereto, and
the vital sign data management server including a first database for the household side and a second database for the facility side and being configured to
assign a unique first database ID to the first database,
assign a unique second database ID to the second database,
create a record for each of the member names on the household side, associate the IDs unique to each of the member names with the records, and register, for each of the records, the household-side record address associated with the ID unique to each of the member names, in the first database,
create a record for each of the member names on the facility side, associate the IDs unique to each of the member names with the records, and register, for each of the records, the household-side record address associated with the ID unique to each of the member names, in the second database,
record new vital sign data, transmitted with specification of the ID unique to each of the member names from the second communication terminal to the second database, in a vital sign data field in the record having the ID unique to each of the member names in the second database, and
when the new vital sign data is recorded in the vital sign data field of the second database, use the household-side record address transmitted along with the new vital sign data from the second communication terminal as a key to identify the ID unique for the member name of the household-side record address registered in the first database, write the same data into the vital sign data field of the record having the ID, and notify the first communication terminal of data writing.

6. The vital sign data management method according to claim 5, wherein
the vital sign data management server, when identifying the ID unique for the member name of the facility-side record address registered in the first database and writing the same data into the vital sign data field of the record having the ID, prohibits, when a time stamp of the data is identical to a time stamp of the vital sign data previously written, the writing of the vital sign data subsequently generated.

7. The vital sign data management system according to claim 6, wherein
the second communication terminal is used on the facility side, such as in a nursery school entrusted with infants or a daycare center entrusted with preschoolers, and
the group name registered in the second database is a name of a class of a facility such as a nursery school or a daycare center, and the member names are names of infants entrusted to the nursery school or names of preschoolers entrusted to the daycare center.

8. The vital sign data management system according to claim 5, wherein
text data and image data can be recorded in the vital sign data field in each record of the first database and the second database.

9. A vital sign data management method of a vital sign data management system including
a plurality of first communication terminals respectively provided on a plurality of households,
a second communication terminal on a facility side, and
a vital sign data management server connected to the plurality of first communication terminals and the second communication terminal via an Internet,
the vital sign data management method comprising:
in the plurality of first communication terminals, by execution of a vital sign data management application installed therein,
receiving input of a group name in units of household and a member name of each member of the household, and generating IDs unique to each of the member names to notify the vital sign data management server thereof, and
associating vital sign data measured or manually input for each of the member names with the ID unique to the member name, and transmitting, to the vital sign data management server via the Internet, the vital sign data along with a facility-side record address informed by the facility side,
in the second communication terminal, by execution of the vital sign data management application installed therein,
receiving input of one or a plurality of group names on the facility side and each member name belonging to each group, and generating IDs unique to each of the member names to notify the vital sign data management server thereof,
issuing, for each of the member names, the facility-side record address unique and corresponding to the ID unique to the member name, and
associating vital sign data measured or manually input for each of the member names with the ID unique to the member name, and transmitting the vital sign data to the vital sign data management server via the Internet, and
in the vital sign data management server,
assigning a unique first database ID to a first database for the household side,
assigning a unique second database ID to a second database for the facility side,
creating a record for each of the member names on the household side, associating the IDs unique to each of the member names with the records, and registering, for each of the records, the facility-side record address associated with the ID unique to each of the member names, in the first database,
creating a record for each of the member names on the facility side, associating the IDs unique to each of the member names with the records, and registering, for each of the records, the facility-side record address associated with the ID unique to each of the member names, in the second database,
recording new vital sign data, transmitted with specification of the ID unique to each of the member names from the first communication terminal to the first database, in a vital sign data field in the record having the ID unique to each of the member names in the first database, and
when the new vital sign data is recorded in the vital sign data field of the first database, using the facility-side record address transmitted along with the new vital sign data from the first communication terminal as a key to identify the ID unique for the member name of the facility-side record address registered in the second database, writing the same data into the vital sign data field of the record having the ID, and notifying the second communication terminal of data writing.

10. The vital sign data management method according to claim 9, wherein
the vital sign data management server, when identifying the ID unique for the member name of the facility-side record address registered in the second database and writing the same data into the vital sign data field of the record having the ID, prohibits, when a time stamp of the data is identical to a time stamp of the vital sign data previously written, the writing of the vital sign data subsequently generated.

11. The vital sign data management method according to claim 10, wherein
the second communication terminal is used on the facility side, such as in a nursery school, a daycare center, a preschool, or a school entrusted with children, and
the group name registered in the second database is a name of a class, and the member names are names of students belonging to the class.

12. The vital sign data management method according to claim 9, wherein
text data and image data can be recorded in the vital sign data field in each record of the first database and the second database.

13. A vital sign data management method of a vital sign data management system including
a plurality of first communication terminals respectively provided in a plurality of household sides,
a second communication terminal on a facility side, and
a vital sign data management server connected to the plurality of first communication terminals and the second communication terminal via an Internet,
the vital sign data management method comprising:
in the plurality of first communication terminals, by execution of a vital sign data management application installed therein,
receiving input of a group name in units of household and a member name of each member of the household, and generating IDs unique to the member names to notify the vital sign data management server thereof,
issuing, for each of the member names, a household-side record address unique and corresponding to the ID unique to the member name, and
associating vital sign data measured or manually input for each of the member names with the ID unique to the member name, and transmitting the vital sign data to the vital sign data management server via the Internet,
in the second communication terminal, by execution of the vital sign data management application installed therein,
receiving input of one or a plurality of group names in a facility and each member name belonging to each group, and generating IDs unique to the member names to notify the vital sign data management server thereof, and
associating vital sign data measured or manually input for each of the member names with the ID unique to the member name, and transmitting, to the vital sign data management server via the Internet, the vital sign data with the household-side record address informed by the household side added thereto, and
in the vital sign data management server,
assigning a unique first database ID to a first database for the household sides,
assigning a unique second database ID to a second database for the facility side,
creating a record for each of the member names on the household side, associating the ID unique to each of the member names with the records, and registering, for each of the records, the household-side record address associated with the ID unique to each of the member names, in the first database,
creating a record for each of the member names on the facility side, associating the ID unique to each of the member names with the records, and registering, for each of the records, the household-side record address associated with the ID unique to each of the member names, in the second database,
recording new vital sign data, transmitted with specification of the ID unique to each of the member names from the second communication terminal to the second database, in a vital sign data field in the record having the ID unique to each of the member names in the second database, and
when the new vital sign data is recorded in the vital sign data field of the second database, using the household-side record address transmitted along with the new vital sign data from the second communication terminal as a key to identify the ID unique for the member name of the household-side record address registered in the first database, writing the same data into the vital sign data field of the record having the ID, and notifying the first communication terminal of data writing.

14. The vital sign data management method according to claim 13, wherein
the vital sign data management server, when identifying the ID unique for the member name of the household-side record address registered in the first database and writing the same data into the vital sign data field of the record having the ID, prohibits, when a time stamp of the data is identical to a time stamp of the vital sign data previously written, the writing of the vital sign data subsequently generated.

15. The vital sign data management method according to claim 14, wherein
the second communication terminal is used on the facility side, such as in a nursery school entrusted with infants or a daycare center entrusted with preschoolers, and
the group name registered in the second database is a name of a class of a facility such as a nursery school or a daycare center, and the member names are names of infants entrusted to the nursery school or names of preschoolers entrusted to the daycare center.

16. The vital sign data management method according to claim 13, wherein
text data and image data can be recorded in the vital sign data field in each record of the first database and the second database.
